(19) United States
(12) Patent Application Publication
(10) Pub. No.: US 2020/0077895 A1
(43) Pub. Date: Mar. 12, 2020

(54) CARDIAC IMAGE PROCESSING APPARATUS, SYSTEM, AND METHOD

(71) Applicant: TERUMO KABUSHIKI KAISHA, Tokyo (JP)

(72) Inventor: Yasuyuki HONMA, Kanagawa (JP)

(21) Appl. No.: 16/681,325

(22) Filed: Nov. 12, 2019

Related U.S. Application Data
(63) Continuation of application No. PCT/JP2018/018901, filed on May 16, 2018.

(30) Foreign Application Priority Data
May 16, 2017 (JP) 2017-097659

(51) Int. Cl. A61B 5/00; A61B 5/042; A61B 5/024; G06T 7/00

(52) U.S. Cl. CPC A61B 5/0044; A61B 5/0035; A61B 5/042; A61B 5/024; G06T 2207/10088; G06T 7/0012; G06T 2207/30048; G06T 2207/10132; G06T 2207/10116; A61B 5/6852

(57) ABSTRACT

An image processing apparatus is described herein including an image input unit that receives an input of a tomographic image of a heart imaged from outside a body; a low motion site estimation unit that estimates a low motion site of the heart on the basis of the tomographic image; an infarct site estimation unit that estimates an infarct site of the heart; and a target site identification unit that identifies a site other than the infarct site among the low motion sites, as a target site.

### CROSS REFERENCE TO RELATED APPLICATIONS

The present application is a continuation of and claims benefit to PCT Application No. PCT/JP2018/018901, filed on May 16, 2018, entitled “IMAGE PROCESSING DEVICE, IMAGE PROCESSING SYSTEM AND IMAGE PROCESSING METHOD” which claims priority to Japanese Patent Application No. 2017-097659, filed on May 16, 2017. The entire disclosures of the applications listed above are hereby incorporated by reference, in their entirety, for all that they teach and for all purposes.

### FIELD

The present disclosure relates to an image processing apparatus, an image processing system, and an image processing method.

### BACKGROUND

There is a current treatment, in the treatment of heart failure or the like, that injects a biological substance such as a cell or an administration substance such as a biomaterial into a tissue for achieving therapeutic effects. In such procedures, instruments such as catheters are used for performing the injection into tissues. In cell therapy using such a catheter or the like, 3D mapping or the like is performed on a biological tissue such as a heart ventricle before the injection procedure and thereby identifies the position of an infarct. Thereafter, cells or the like as an administration substance may be injected and directed to a desired position according to the treatment, such as a boundary between the infarct and the normal myocardial tissue. For example, Japanese Patent Application No. JP 2009-106530 A describes that a site having low heart wall motion may be estimated as an abnormal site from an ultrasound image or the like, so as to create a diagnostic image.

### SUMMARY

### Technical Problem

However, while the technology described in Japanese Patent Application No. JP 2009-106530 A can estimate the site having low heart wall motion as an abnormal site, it has not been sufficient to identify the site having low wall motion from the viewpoint of therapeutic effects.

In view of the above problems, an object of the present disclosure is to provide an image processing apparatus, an image processing system, and an image processing method capable of contributing to improvement in therapeutic effects.

### Solution to the Problem

An image processing apparatus according to a first aspect of the present disclosure includes: an image input unit that receives as an input a tomographic image of a heart taken from outside a body; a low motion site estimation unit that estimates a low motion site of the heart on the basis of the tomographic image; an infarct site estimation unit that estimates an infarct site of the heart; and a target site identification unit that identifies a site other than the infarct site among the low motion sites, as a target site, the target site displayed on an output of the tomographic image.

In the image processing apparatus according to an embodiment of the present disclosure, the infarct site estimation unit acquires electrocardiographic information indicating an electrocardiogram of a heart wall with which a distal end portion of a catheter comes in contact via an electrode provided on the distal end portion of the catheter, and estimates the infarct site on the basis of the acquired electrocardiographic information.

In the image processing apparatus according to an embodiment of the present disclosure, the infarct site estimation unit acquires electrocardiographic information indicating an electrocardiogram of a heart wall on the basis of a captured image obtained by imaging the heart by a predetermined imaging device, and estimates the infarct site on the basis of the acquired electrocardiographic information.

In the image processing apparatus according to an embodiment of the present disclosure, when the tomographic image is a first tomographic image, the image input unit further receives an input of a second tomographic image of the heart taken from outside the body, and the infarct site estimation unit estimates the infarct site on the basis of the second tomographic image.

In the image processing apparatus according to an embodiment of the present disclosure, the image input unit receives an input of a plurality of first tomographic images captured every predetermined time, and the low motion site estimation unit estimates the low motion site on the basis of temporal changes in the plurality of first tomographic images.

The image processing apparatus according to an embodiment of the present disclosure further includes: a feature point detection unit that detects a feature point from each of the first tomographic image and the second tomographic image; and an expansion/contraction state estimation unit that estimates an expansion/contraction state of the heart in each of the first tomographic image and the second tomographic image on the basis of position information of the feature point.

The image processing apparatus according to an embodiment of the present disclosure further includes: a heart rate input unit that receives an input of heart beat information; and an expansion/contraction state estimation unit that estimates an expansion/contraction state of the heart in each of the first tomographic image and the second tomographic image on the basis of the heart beat information.

The image processing apparatus according to an embodiment of the present disclosure further includes a display information generation unit that generates display information in which the target site is superimposed on one of the first tomographic image or the second tomographic image.

In the image processing apparatus according to an embodiment of the present disclosure, the display information generation unit generates the display information by correcting the first tomographic image on the basis of the second tomographic image.

In the image processing apparatus according to an embodiment of the present disclosure, the first tomographic image is an ultrasound image.

In the image processing apparatus according to an embodiment of the present disclosure, the second tomographic image includes a delayed contrast-enhanced image, and the infarct site estimation unit estimates the infarct site on the basis of the delayed contrast-enhanced image.

In the image processing apparatus according to an embodiment of the present disclosure, the second tomographic image is one of a radiological image or a magnetic resonance image.

An image processing system as a second aspect of the present disclosure includes an imaging device that captures a tomographic image of a heart from outside the body, and an image processing apparatus, in which the image processing apparatus includes: an image input unit that receives an input of the tomographic image; a low motion site estimation unit that estimates a low motion site of the heart on the basis of the tomographic image; an infarct site estimation unit that estimates an infarct site of the heart; and a target site identification unit that identifies a site other than the infarct site among the low motion sites, as a target site.

An image processing method as a third aspect of the present disclosure is an image processing method executed using an image processing apparatus, the method including: an image input step of receiving as an input a tomographic image of a heart taken from outside the body; a low motion site estimation step of estimating a low motion site of the heart on the basis of the tomographic image; an infarct site estimation step of estimating an infarct site of the heart; and a target site identification step of identifying a site other than the infarct site among the low motion sites, as a target site, the target site displayed on an output of the tomographic image.

### Non-Exhaustive Advantages

According to the image processing apparatus, the image processing system, and the image processing method of the present disclosure, it is possible to contribute to an improvement in therapeutic effects.

### BRIEF DESCRIPTION OF THE DRAWINGS

FIG. 1 is a schematic block diagram illustrating an image processing system including an image processing apparatus in accordance with embodiments of the present disclosure.

FIG. 2 is a flowchart illustrating a method for performing image processing by the image processing apparatus illustrated in FIG. 1.

FIG. 3 is a flowchart illustrating details of target site identification processing performed by the image processing apparatus illustrated in FIG. 1.

FIG. 4A is a schematic view illustrating image processing of a first tomographic input accompanying target site identification processing performed by the image processing apparatus illustrated in FIG. 1.

FIG. 4B is a schematic view illustrating image processing of a second tomographic input accompanying target site identification processing performed by the image processing apparatus illustrated in FIG. 1.

FIG. 4C is a schematic view illustrating image processing where an abnormal site of the heart is identified by the image processing apparatus illustrated in FIG. 1.

FIG. 5 is a schematic view illustrating an example of a permeation region estimated by a permeation region estimation processing performed by the image processing apparatus illustrated in FIG. 1.

FIG. 6 is a flowchart illustrating details of target injection point determination processing performed by the image processing apparatus illustrated in FIG. 1.

FIG. 7A is a first schematic view illustrating an example of a target injection point determined by a target injection point determination processing performed by the image processing apparatus illustrated in FIG. 1.

FIG. 7B is a second schematic view illustrating an example of a target injection point determined by a target injection point determination processing performed by the image processing apparatus illustrated in FIG. 1.

FIG. 8 is a schematic view illustrating a state of treatment with an injection member in accordance with embodiments of the present disclosure.

### DETAILED DESCRIPTION

Hereinafter, an embodiment of the present disclosure will be described with reference to the drawings. In the drawings, common members are denoted by the same reference numerals.

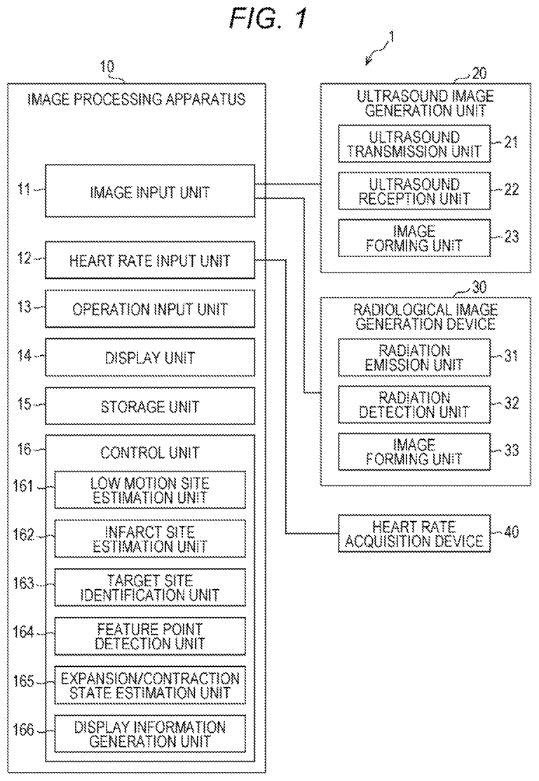

FIG. 1 is a block diagram illustrating a schematic configuration of an image processing system 1 including an image processing apparatus 10 as one embodiment of the present disclosure. As illustrated in FIG. 1, the image processing system 1 of the present embodiment includes an image processing apparatus 10, an ultrasound image generation unit, or device, 20 as a first imaging device, and a radiological image generation device 30 as a second imaging device, and a heart rate acquisition device 40.

The ultrasound image generation device 20 as the first imaging device is located outside the body of the subject and captures an ultrasound image as a first tomographic image of the heart from outside the subject's body. The ultrasound image generation device 20 includes an ultrasound transmission unit 21 that transmits ultrasounds, an ultrasound reception unit 22 that receives ultrasounds, and an image forming unit 23 that forms a first tomographic image on the basis of the ultrasounds received by the ultrasound reception unit 22. The ultrasound image generation device 20 transmits ultrasounds from the ultrasound transmission unit 21 toward the subject's heart in a state where the ultrasound transmission unit 21 and the ultrasound reception unit 22 are in contact with the body surface of the subject, and receives the ultrasound reflected from the heart of the subject, on the ultrasound reception unit 22. The ultrasound image generation device 20 processes, on the image forming unit 23, the ultrasound received by the ultrasound reception unit 22, and thereby obtains a tomographic image along a traveling plane of the ultrasound, as a first tomographic image. The ultrasound image generation device 20 outputs the captured first tomographic image to the image input unit 11 of the image processing apparatus 10.

The ultrasound image generation device 20 may generate a three-dimensional image as the first tomographic image on the basis of a plurality of tomographic images captured along various planes by changing position or orientation of the ultrasound transmission unit 21 and the ultrasound reception unit 22. That is, the first tomographic image may be a tomographic image captured along one plane, or a three-dimensional image generated on the basis of a plurality of tomographic images taken along a plurality of planes.

The radiological image generation device 30 as the second imaging device is located outside the body of the subject and captures a radiological image as a second tomographic image of the heart from outside the subject's body. The radiological image generation device 30 is implemented as a computed tomography (CT) device, for example. The radiological image generation device 30 includes a radiation emission unit 31 that emits radiation, a radiation detection unit 32 that detects radiation, and an image forming unit 33 that forms a second tomographic image on the basis of the radiation detected by the radiation detection unit 32. The radiological image generation device 30 includes a radiation emission unit 31 and a radiation detection unit 32 at positions facing each other around the subject. Radiation, such as X-rays, may be emitted from the radiation emission unit 31 toward the subject's heart while rotating the radiation emission unit 31 and the radiation detection unit 32 around the subject, and the radiation that has passed through the subject's heart is detected by the radiation detection unit 32. The radiological image generation device 30 processes, in the image forming unit 33, the radiation detected by the radiation detection unit 32 and thereby obtains a radiological image that is a three-dimensional image of the heart, as a second tomographic image. The radiological image generation device 30 outputs the captured second tomographic image to the image input unit 11 of the image processing apparatus 10.

The second imaging device may be a magnetic resonance imaging (MRI) device instead of the radiological image generation device 30. The magnetic resonance image generation device is located outside the subject's body and captures a magnetic resonance image as a second tomographic image of the heart from outside the subject's body. The magnetic resonance image generation device includes a magnetic field generation unit that generates a magnetic field, a signal reception unit that receives a nuclear magnetic resonance signal, and an image forming unit that forms a magnetic resonance image being a three-dimensional image, as a second tomographic image, on the basis of the nuclear magnetic resonance signal received by the signal reception unit.

A contrast agent is administered to the subject's heart a predetermined time before the second tomographic image is captured by the radiological image generation device 30 as the second imaging device or the magnetic resonance image generation device. Thereby, the second tomographic image captured by the second imaging device includes a delayed contrast-enhanced image.

The second imaging device may be a radio isotope inspection device that performs scintigraphy inspection, Single Photon Emission Computed Tomography (SPECT) inspection, Positron Emission Tomography (PET) inspection, or the like instead of the radiological image generation device 30 or the magnetic resonance image generation device. The radio isotope inspection device is located outside the body of the subject and acquires a radioisotope (RI) distribution image as a second tomographic image of the heart from outside the subject's body. The radio isotope inspection device acquires the second tomographic image by imaging the distribution of the agent labeled with the radioisotope previously administered to the subject.

The heart rate acquisition device 40 acquires cardiac heartbeat information of the subject. The heartbeat information includes temporal change information in the heartbeat. The heart rate acquisition device 40 may acquire the heartbeat information simultaneously as the first tomographic image or the second tomographic image, and may associate the heartbeat information with the image. The heart rate acquisition device 40 is, for example, an electrocardiogram monitor that measures temporal changes in cardiac action potential via electrodes attached to the subject's chest or limbs and continuously displays the electrocardiogram waveform over time.

The image processing apparatus 10 is located outside the body of the subject and is implemented by an information processing device such as a computer. The image processing apparatus 10 includes an image input unit 11, a heart rate input unit 12, an operation input unit 13, a display unit 14, a storage unit 15, and a control unit 16.

The image input unit 11 receives an input of a first image from the ultrasound image generation device 20 as the first imaging device. The image input unit 11 receives an input of the second image from the radiological image generation device 30 as the second imaging device. The image input unit 11 includes an interface that receives information from the ultrasound image generation device 20 and the radiological image generation device 30 by wired communication or wireless communication, for example. The image input unit 11 outputs information regarding the input image to the control unit 16.

The heart rate input unit 12 receives an input of heartbeat information from the heart rate acquisition device 40. The heart rate input unit 12 includes an interface that receives information from the heart rate acquisition device 40 by wired communication or wireless communication, for example. The heart rate input unit 12 outputs the input heartbeat information to the control unit 16.

The operation input unit 13 includes a keyboard, a mouse, or a touch panel, for example. In a case where the operation input unit 13 includes a touch panel, the touch panel may be provided integrally with the display unit 14. The operation input unit 13 outputs the input information to the control unit 16.

The display unit 14 displays (e.g., renders images, etc.), on the basis of a signal from the control unit 16, the first tomographic image, the second tomographic image, and an image generated by the control unit 16 on the basis of these images. The display unit 14 includes a display device such as a liquid crystal display or an organic electroluminescent (EL) display, for example.

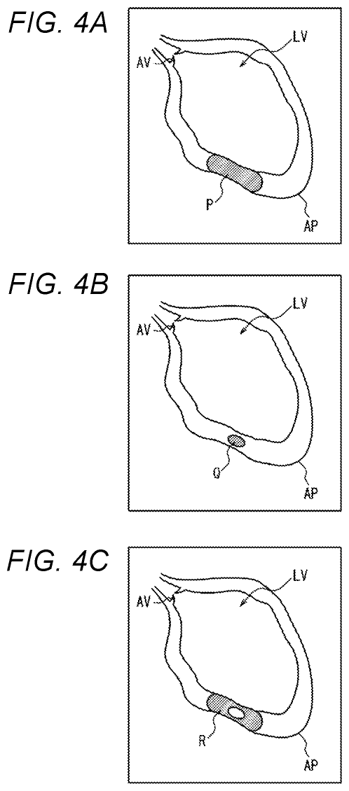

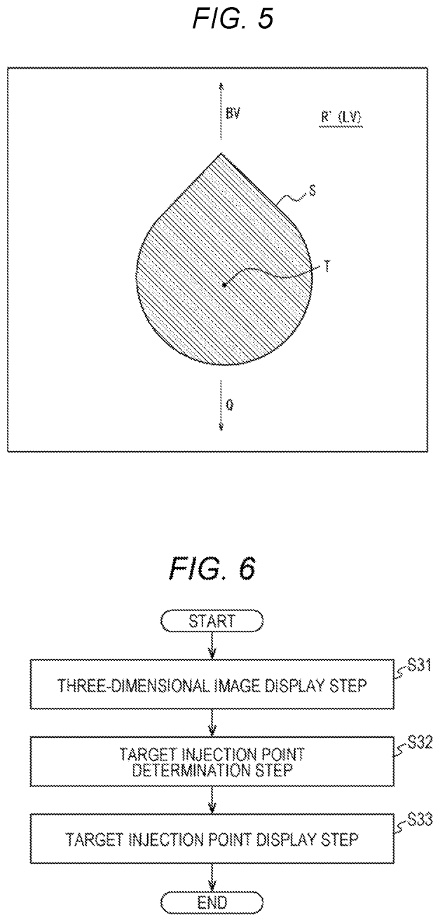

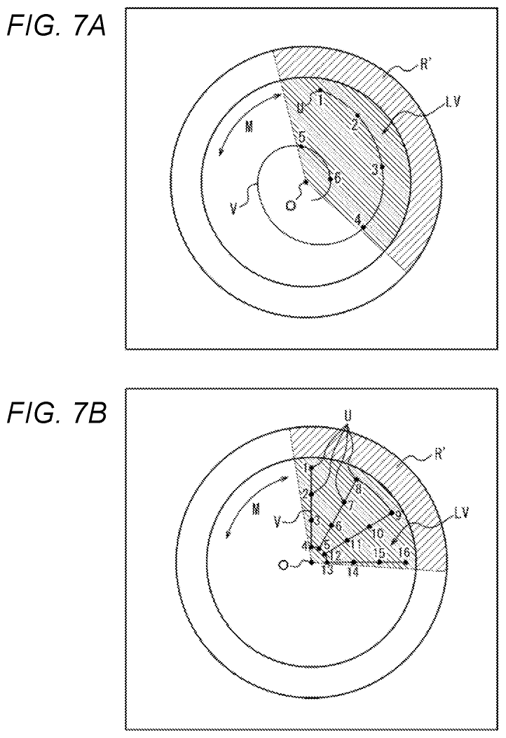

The storage unit 15 stores various types of information and programs for causing the control unit 16 to execute specific functions. The storage unit 15 stores a three-dimensional image of the heart, for example. The three-dimensional image of the heart is the first tomographic image, the second tomographic image, or display information generated by the control unit 16 on the basis of these images by target site identification processing described below. The three-dimensional image of the heart includes an abnormal site R′ (refer to FIGS. 5 and 7A-7B) of the heart. The abnormal site R′ of the heart is, for example, a target site R (refer to FIG. 4C) identified by the control unit 16 in a target site identification processing described below. The storage unit 15 stores a plurality of three-dimensional images based on a plurality of tomographic images captured at different times, for example. The storage unit 15 stores administration dose and physical property information of the administration substance to be injected into the abnormal site R′ by treatment using an injection member to be described below, for example. The storage unit 15 stores shape information of the injection member, for example. The storage unit 15 includes a storage device such as a random-access memory (RAM) or a read-only memory (ROM), for example.

The control unit 16 controls operation of each of components of the image processing apparatus 10. The control unit 16 executes a specific function by reading a specific program. Specifically, the control unit 16 generates display information on the basis of the first tomographic image and the second tomographic image. The control unit 16 causes the display unit 14 to display the generated display information. The control unit 16 may output the generated display information to an external display device. The control unit 16 includes a processor, for example.

The control unit 16 includes a low motion site estimation unit 161, an infarct site estimation unit 162, a target site identification unit 163, a feature point detection unit 164, an expansion/contraction state estimation unit 165, and a display information generation unit 166.

The low motion site estimation unit 161 estimates a low motion site of the heart on the basis of the first tomographic image of the heart input via the image input unit 11. The infarct site estimation unit 162 estimates an infarct site of the heart on the basis of the second tomographic image of the heart input via the image input unit 11. The target site identification unit 163 identifies a site other than the infarcted site among the low motion sites, as a target site. The feature point detection unit 164 detects a feature point from each of the first tomographic image and the second tomographic image. The expansion/contraction state estimation unit 165 estimates the expansion/contraction state of the heart in each of the first tomographic image and the second tomographic image. The display information generation unit 166 generates display information on the basis of the first tomographic image and the second tomographic image. The display information generation unit 166 generates display information in which the target site is superimposed on the first tomographic image or the second tomographic image, for example.

In a case where the second tomographic image is captured by the radiological image generation device 30 or the magnetic resonance image generation device, the display information generation unit 166 may generate display information by correcting the first tomographic image on the basis of the second tomographic image. For example, the feature point detection unit 164 detects the feature point in the first tomographic image and the feature point in the second tomographic image by pattern recognition or the like, and the display information generation unit 166 replaces the region including the feature point in the first tomographic image with a region within the second tomographic image including the corresponding feature point, making it possible to generate display information obtained by correcting the first tomographic image on the basis of the second tomographic image. With this configuration, the first tomographic image can be corrected with a higher-definition second tomographic image, making it possible to further correctly demonstrate the structure and shape information of the heart.

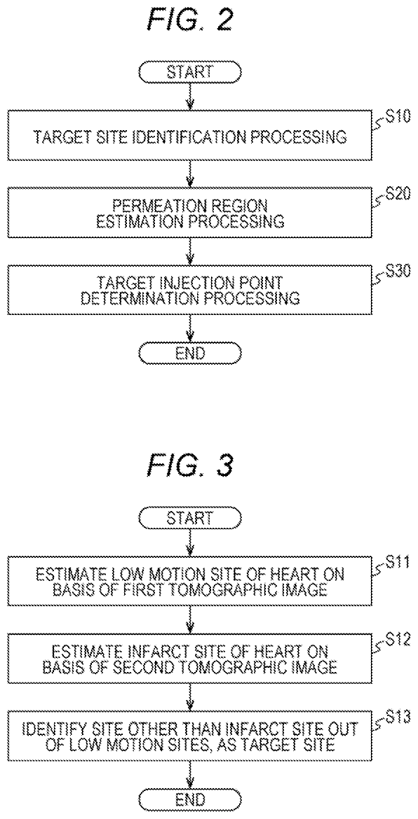

FIG. 2 is a flowchart illustrating a method of image processing performed by the image processing apparatus 10. As illustrated in FIG. 2, the image processing apparatus 10 first performs target site identification processing (step S10). Next, the image processing apparatus 10 performs permeation region estimation processing (step S20). Finally, the image processing apparatus 10 performs target injection point determination processing (step S30).

FIG. 3 is a flowchart illustrating details of target site identification processing performed by the image processing apparatus 10. FIGS. 4A-4C are views illustrating image processing accompanying the target site identification processing performed by the image processing apparatus 10, and illustrating a cross section of a left ventricle LV of the heart. As illustrated in FIG. 4A, the low motion site estimation unit 161 of the image processing apparatus 10 reads the first tomographic image input via the image input unit 11, and estimates a low motion site P of the heart on the basis of the first tomographic image (step S11: low motion site estimation step). Specifically, the image input unit 11 receives an input of a plurality of first tomographic images captured at predetermined times. The low motion site estimation unit 161 estimates the low motion site P on the basis of the temporal change of the plurality of first tomographic images. More specifically, the feature point detection unit 164 first extracts a plurality of points having luminance of a predetermined value or more in the first tomographic image, as feature points. The feature point detection unit 164 extracts a plurality of feature points from each of a plurality of first tomographic images captured at different times including the diastole in which the myocardium is most dilated and the systole in which the myocardium is most deflated. The display information generation unit 166 calculates a change rate obtained by measuring the distance between an arbitrary feature point and another adjacent feature point in the first tomographic image in the diastole and the first tomographic image in the systole, and then the calculated change rate is reflected onto the three-dimensional image of the heart. For example, the display information generation unit 166 generates a three-dimensional image of the heart so that a region where the change rate is a predetermined threshold or less and a region where the change rate exceeds a predetermined threshold are in different modes (for example, rendered in different colors, etc.). The low motion site estimation unit 161 estimates that the site of the heart corresponding to the region in which the change rate is a predetermined threshold or less is the low motion site P. The predetermined threshold of the change rate is, for example, 12%, but may be appropriately altered by setting.

As illustrated in FIG. 4B, the infarct site estimation unit 162 reads the second tomographic image input via the image input unit 11, and estimates an infarct site Q of the heart on the basis of the second tomographic image (step S12: infarct site estimation step). The infarct site Q is a site where the myocardium is ischemic and necrotic. The infarct site Q is a site where the above change rate is a predetermined threshold or less and is included in the low motion site P. Specifically, in a case where the second tomographic image includes a delayed contrast-enhanced image, the infarct site estimation unit 162 estimates the infarct site Q on the basis of the delayed contrast-enhanced image of the second tomographic image. Specifically, the infarct site estimation unit 162 estimates the site in which the delayed contrast-enhanced image is imaged as the infarct site Q. In a case where the second tomographic image is a radioisotope distribution image, the infarct site estimation unit 162 estimates the infarct site Q on the basis of the radioisotope distribution. Specifically, the infarct site estimation unit 162 estimates the accumulated defect site where radioisotopes are not accumulated as the infarct site Q. The infarct site estimation unit 162 may execute the infarct site estimation step (step S12) prior to the low motion site estimation step (step S11) by the low motion site estimation unit 161 or the like described above.

As illustrated in FIG. 4C, the target site identification unit 163 identifies the site other than the infarct site Q estimated in the infarct site estimation step (step S12) out of the low motion sites P estimated in the low motion site estimation step (step S11), as the target site R (step S13: target site identification step). The target site R is a site where the change rate is a predetermined threshold or less but is not necrotic, which is a hibernating myocardium or a stunned myocardium. The display information generation unit 166 generates display information in which the identified target site R is superimposed on the first tomographic image or the second tomographic image. The target site R includes the hibernating myocardium and the stunned myocardium, each of which exists independently of each other. The hibernating myocardium is a chronic ischemic state and the stunned myocardium is an acute ischemic state. Stunned myocardium is caused by overload due to reperfusion. Therefore, the site of stunned myocardium can be identified by generating an overload condition and then eliminating the overload condition. This makes it possible to select stunned myocardium and hibernating myocardium.

Since the heart repeatedly contracts and dilates with heartbeat, it would be preferable that an expansion/contraction state of the heart in the first tomographic image used in the low motion site estimation step (step S11) and the expansion/contraction state of the heart in the second tomographic image used in the infarct site estimation step (step S12) are in a same or similar state. Therefore, the target site identification unit 163 selects a first tomographic image corresponding to the expansion/contraction state of the heart in the second tomographic image, from among the plurality of first tomographic images, and uses the selected first tomographic image to identify the target site R. The expansion/contraction state of the heart in the first tomographic image may be estimated on the basis of position information of a feature point detected from the first tomographic image by pattern recognition or the like using the feature point detection unit 164. Similarly the expansion/contraction state of the heart in the second tomographic image may be estimated on the basis of position information of a feature point detected from the second tomographic image by pattern recognition or the like using the feature point detection unit 164. The feature points include, for example, an apex AP or an aortic valve AV. The expansion/contraction state of the heart in the first tomographic image and the second tomographic image may be estimated on the basis of the heart beat information input via the heart rate input unit 12. Specifically, the first tomographic image and the second tomographic image are associated with heartbeat information at the time of imaging, and the expansion/contraction state of the heart in the first tomographic image and the second tomographic image is estimated by individually associated heartbeat information.

As described above, the image processing apparatus 10 can identify hibernating myocardium or stunned myocardium having a relatively high therapeutic effect as the target site R, making it possible to contribute to an improvement in therapeutic effects.

The method by which the infarct site estimation unit 162 estimates the infarct site of the heart is not limited to the method described above. The infarct site estimation unit 162 can estimate the infarct site on the basis of electrocardiographic information indicating the cardiac potential of the heart wall, for example. In general, it is known that the cardiac potential is less than 7.0 mV at the infarct site, while the cardiac potential is 7.0 mV or more at the normal site and the hibernating myocardium. Therefore, a site where the cardiac potential is less than a predetermined threshold (for example, less than 7.0 mV) can be estimated as an infarct site.

There are various methods for acquiring electrocardiographic information. For example, methods for acquiring electrocardiographic information may include a method in which an electrode is provided at a distal end portion of a catheter, and the distal end portion of the catheter is brought into contact with the heart wall and thereby acquires, via the electrode, electrocardiographic information of the heart wall with which the distal end portion of the catheter comes in contact. Moreover, there is another method using a captured image obtained by imaging the heart using predetermined imaging devices such as an ultrasound diagnostic device, an X-ray CT device, or an MRI device. This method utilizes a link between electrical excitation of the myocardium and contraction of the myocardium, and acquires electrocardiographic information on the basis of a captured image obtained by imaging the heart with a predetermined imaging device (various imaging devices described above). Specifically, electrocardiographic information can be acquired from the pattern of contraction propagation due to wall motion observed in the captured image. The predetermined imaging device to be used may be the above-described ultrasound image generation device 20 (refer to FIG. 1) or radiological image generation device 30 (refer to FIG. 1).

FIG. 5 is a schematic view illustrating an example of a permeation region S estimated by permeation region estimation processing performed by the image processing apparatus 10. FIG. 5 is a view illustrating a cross section of the heart wall of the left ventricle LV of the heart, and illustrates a range of the permeation region S located in an abnormal site R′. When it is assumed that the administration substance is injected at an arbitrary injection point T of the abnormal site R′ included in a three-dimensional image of the heart stored in the storage unit 15, the control unit 16 estimates the permeation region S at which the administration substance would permeate (permeation region estimation step). The control unit 16 generates display information in which the estimated permeation region S is superimposed on the three-dimensional image. The abnormal site R′ of the heart is the target site R identified by the above-described target site identification processing, for example. The administration substance is a biological substance such as a cell or a substance such as a biomaterial, for example. The permeation region S is a region after a predetermined time has elapsed within a time period during which the effect of the administration substance is obtained, from the time the administration substance is injected.

For example, the control unit 16 estimates the position of the blood vessel BV in the heart on the basis of a three-dimensional image, and estimates the permeation region S on the basis of the position of the injection point T with respect to the position of the blood vessel BV. The administration substance injected into the abnormal site R′ is considered to easily permeate in the direction of the blood vessel BV due to the influence of blood flow, near the blood vessel BV. Therefore, as illustrated in FIG. 5, the control unit 16 estimates that closer the injection point T is to the blood vessel BV, the more the permeation region S extends in the direction of the blood vessel BV For example, the control unit 16 estimates the position of the infarct site Q on the basis of a three-dimensional image, and estimates the permeation region S on the basis of the position of the injection point T with respect to the position of the infarct site Q. It is considered that the administration substance injected into the abnormal site R′ is less likely to permeate in the direction of the infarct site Q because the heart activity such as blood flow or heart beat is reduced near the infarct site Q, for example. Therefore, as illustrated in FIG. 5, the control unit 16 estimates that the closer the injection point T is to the infarct site Q, the more the permeation region S is prevented from extending in the direction of the infarct site Q.

The control unit 16 may estimate the permeation region S on the basis of the administration dose and physical property information of the administration substance stored in the storage unit 15. Specifically, the control unit 16 estimates that the more the administration dose of the administration substance, the larger the permeation region S is. The control unit 16 may estimate the wall thickness for each of sites of the heart on the basis of the three-dimensional image, and may estimate the permeation region S on the basis of the wall thickness. Specifically, the control unit 16 estimates that the thinner the wall thickness near the injection point T is, the wider the permeation region S becomes along the heart wall. The control unit 16 may estimate the permeation region S on the basis of temporal change of a plurality of three-dimensional images stored in the storage unit 15. Specifically, the control unit 16 detects a temporal change in the positions of feature points in a plurality of three-dimensional images, and estimates the motion due to heartbeat or the like for each of sites of the heart wall on the basis of the temporal change in the positions of the feature points. Subsequently the control unit 16 estimates that the greater the motion of the site, the larger the permeation region S becomes. The control unit 16 may estimate the permeation region S on the basis of the shape information of the injection member stored in the storage unit 15. The injection member is formed of a needle-like member, with a side hole for discharging the administration substance formed around the injection member, for example. Examples of the shape information of the injection member include the outer shape (linear shape, curved shape, spiral shape, etc.), diameter, side hole position, side hole size, or the like, of the injection member.

As described above, the image processing apparatus 10 can preliminarily estimate the permeation region S into which the administration substance injected at an arbitrary injection point T of the abnormal site R′ would permeate, making it possible to perform therapeutic simulation before performing actual therapy.

FIG. 6 is a flowchart illustrating details of target injection point determination processing performed by the image processing apparatus 10. FIG. 7 is a schematic view illustrating an example of a target injection point U determined by the target injection point determination processing performed by the image processing apparatus 10. FIGS. 7A-7B are a cross-sectional views of the left ventricle LV of the heart as viewed from the aortic valve AV (refer to FIGS. 4A-4C) in the direction of the apex AP (refer to FIGS. 4A-4C). The control unit 16 reads out a three-dimensional image stored in the storage unit 15 and causes the display unit 14 to display the image (step S31: three-dimensional image display step). On the basis of the three-dimensional image, the control unit 16 determines the positions of a plurality of target injection points U at which the administration substance should be injected into the abnormal site R′ (step S32: target injection point determination step). The control unit 16 causes the display unit 14 to display the determined plurality of target injection points U to be superimposed on the three-dimensional image (step S33: target injection point display step). The position of the target injection point U includes information about the depth along the wall thickness direction from the inner surface of the heart wall. In other words, the target injection point U indicates at what position from the inner surface of the heart wall and at what depth the administration substance should be injected. The position of the target injection point U is determined on the basis of the permeation region S estimated by the above-described permeation region estimation processing, for example. Specifically, the control unit 16 estimates the permeation regions S for each of the plurality of injection points T, and determines the injection point T at which the administration substance is to be injected, as the target injection point U on the basis of the estimated plurality of permeation regions S. For example, the control unit 16 identifies the injection point T corresponding to the permeation region S included in the other plurality of permeation regions S. Subsequently, an injection point T other than the specified injection point T is determined as the target injection point U. With this processing, injecting the administration substance at the target injection point U can cause the permeation region S with the administration substance injected at the target injection point U to fill the abnormal site R′ more efficiently.

The control unit 16 determines the order of the plurality of target injection points U. The control unit 16 causes the display unit 14 to display a plurality of target injection points U in a manner based on the determined order. For example, as illustrated in FIG. 7, the control unit 16 performs control such that the determined order is to be written together with the target injection point U. For example, the control unit 16 performs control to display only the target injection point U in the next order. The control unit 16 estimates a movement path V in which the distal end portion of the injection member for injecting the administration substance moves via the plurality of target injection points U, and determines the order of the target injection points U on the basis of the movement path V. For example, the control unit 16 determines the order of the target injection points U so as to minimize the movement path V. Specifically, the control unit 16 determines the order of the target injection points U so as to be closest to each other. The control unit 16 may cause the display unit 14 to display the estimated movement path V to be superimposed on the three-dimensional image. Thereby, an operator such as a medical worker can grasp the optimum way of moving the injection member according to the order of the target injection points U.

As illustrated in FIG. 7A, the control unit 16 may determine the order of the target injection points U so that the movement path V draws a spiral around a major axis O from the aortic valve AV (refer to FIGS. 4A-4C) directed to the apex AP (refer to FIGS. 4A-4C) in the left ventricle LV of the heart. This would set the movement path V as a path that travels in the left ventricle LV along a circumferential direction M from the front aortic valve side toward the back apex side without returning in the middle, making it possible to facilitate operation of the injection member.

As illustrated in FIG. 7B, the control unit 16 may determine the order of the target injection points U so that the movement path V reciprocates along the major axis O from the aortic valve AV toward the apex AP in the left ventricle LV of the heart. With this configuration, the movement path V runs along the major axis O, making it possible to reduce the possibility that the movement of the injection member is hindered by the papillary muscle located along the major axis O in the left ventricle LV, leading to reduction of trapping on the chordae tendineae accompanying the mitral valve.

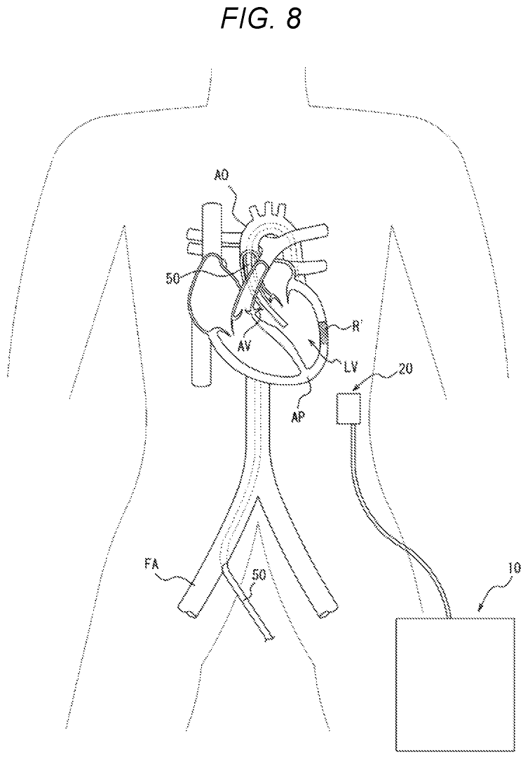

FIG. 8 is a view illustrating a state of treatment by the injection member. FIG. 8 illustrates a state where a catheter 50 extends from a femoral artery FA through the aorta AO to the aortic valve AV which is an entrance of the left ventricle LV of the cardiac lumen. The injection member is delivered through the catheter 50 to the left ventricle LV. The catheter 50 may extend not only from the femoral artery FA, and may extend from the radial artery of the wrist to the aortic valve AV, for example.

As illustrated in FIG. 8, the ultrasound image generation device 20 is located on a body surface of the subject, captures a first tomographic image as necessary, and transmits the captured image to the image processing apparatus 10. The ultrasound image generation device 20 acquires the position information of the distal end portion of the injection member as necessary, and transmits the acquired information to the image processing apparatus 10. With this configuration, the control unit 16 of the image processing apparatus 10 can cause the display unit 14 to display a three-dimensional image following the position of the distal end portion of the injection member, as display information. The ultrasound image generation device 20 may perform imaging not merely from the body surface but also from the esophagus, blood vessel, and cardiac lumen (atrium, ventricle). Still, it is preferable that the ultrasound image generation device 20 captures images from the body surface in that non-invasive treatment can be performed.

The control unit 16 may cause the display unit 14 to display the target injection point U that has undergone the injection treatment of the administration substance by the injection member among the plurality of target injection points U in a manner different from the case of the untreated target injection point U. The control unit 16 determines that the target injection point U has undergone the treatment on the basis of an input of a signal indicating that treatment has been completed via the operation input unit 13, for example. The control unit 16 may discriminate the target injection point U that has undergone treatment on the basis of a newly input first tomographic image.

As described above, the image processing apparatus 10 can determine the positions of the plurality of target injection points U used to inject the administration substance into the abnormal site R′, making it possible to perform more specific treatment simulation before performing treatment. The image processing apparatus 10 displays the target injection point U in a manner based on the order in which treatment should be performed, making it possible to give the operator guidance for the treatment in a predetermined order.

The present disclosure is not limited to the configuration specified in each of the above-described embodiments, and various modifications can be made without departing from the description in the claims. For example, the functions included in each of components or steps or the like can be rearranged in a range that causes no logical contradiction, and a plurality of components, steps or the like can be incorporated or further divided.

The present disclosure relates to an image processing apparatus, an image processing system, and an image processing method.

### DESCRIPTION OF REFERENCE CHARACTERS

1 Image processing system
10 Image processing apparatus
11 Image input unit
12 Heart rate input unit
13 Operation input unit
14 Display unit
15 Storage unit
16 Control unit
161 Low motion site estimation unit
162 Infarct site estimation unit
163 Target site identification unit
164 Feature point detection unit
165 Expansion/contraction state estimation unit
166 Display information generation unit
20 Ultrasound image generation device (first imaging device)
21 Ultrasound transmission unit
22 Ultrasound reception unit
23 Image forming unit
30 Radiological image generation device (second imaging device)
31 Radiation emission unit
32 Radiation detection unit
33 Image forming unit
40 Heart rate acquisition device
50 Catheter
AO Aorta
AP Apex
AV Aortic valve
BV Blood vessel
FA Femoral artery
LV Left ventricle
M Circumferential direction
O Major axis
P Low motion site
Q Infarct site
R Target site
R′ Abnormal site
S Permeation region
T Injection point
U Target injection point
V Movement path

What is claimed is:

1. An image processing apparatus comprising:
an image input unit that receives as an input a tomographic image of a heart taken from outside a body;
a low motion site estimation unit that estimates a low motion site of the heart on the basis of the tomographic image;
an infarct site estimation unit that estimates an infarct site of the heart; and
a target site identification unit that identifies a site other than the infarct site among the low motion sites, as a target site, the target site displayed on an output of the tomographic image.

2. The image processing apparatus of claim 1, wherein the infarct site estimation unit acquires electrocardiographic information indicating an electrocardiogram of a heart wall with which a distal end portion of a catheter comes in contact via an electrode provided on the distal end portion of the catheter, and estimates the infarct site on the basis of the acquired electrocardiographic information.

3. The image processing apparatus of claim 1, wherein the infarct site estimation unit acquires electrocardiographic information indicating an electrocardiogram of a heart wall on the basis of a captured image obtained by imaging the heart by a predetermined imaging device, and estimates the infarct site on the basis of the acquired electrocardiographic information.

4. The image processing apparatus of claim 1,
wherein, when the tomographic image is a first tomographic image, the image input unit further receives an input of a second tomographic image of the heart taken from outside the body, and
the infarct site estimation unit estimates the infarct site on the basis of the second tomographic image.

5. The image processing apparatus of claim 4,
wherein the image input unit receives an input of a plurality of first tomographic images captured every predetermined time, and
the low motion site estimation unit estimates the low motion site on the basis of temporal changes in the plurality of first tomographic images.

6. The image processing apparatus of claim 5, wherein the target site identification unit selects a first tomographic image corresponding to an expansion/contraction state of the heart in the second tomographic image from among the plurality of first tomographic images, and identifies the target site using the selected first tomographic image.

7. The image processing apparatus of claim 6, further comprising:
a feature point detection unit that detects a feature point from each of the first tomographic image and the second tomographic image; and
an expansion/contraction state estimation unit that estimates an expansion/contraction state of the heart in each of the first tomographic image and the second tomographic image on the basis of position information of the feature point.

8. The image processing apparatus of claim 6, further comprising:
a heart rate input unit that receives an input of heart beat information; and
an expansion/contraction state estimation unit that estimates an expansion/contraction state of the heart in each of the first tomographic image and the second tomographic image on the basis of the heart beat information.

9. The image processing apparatus of claim 4, further comprising:
a display information generation unit that generates display information in which the target site is superimposed on one of the first tomographic image or the second tomographic image.

10. The image processing apparatus of claim 9, wherein the display information generation unit generates the display information by correcting the first tomographic image on the basis of the second tomographic image.

11. The image processing apparatus of claim 4, wherein the first tomographic image is an ultrasound image.

12. The image processing apparatus of claim 4,
wherein the second tomographic image includes a delayed contrast-enhanced image, and
the infarct site estimation unit estimates the infarct site on the basis of the delayed contrast-enhanced image.

13. The image processing apparatus of claim 4, wherein the second tomographic image is one of a radiological image or a magnetic resonance image.

14. An image processing system comprising:
an imaging device that captures a tomographic image of a heart from outside a body; and
an image processing apparatus, comprising:
an image input unit that receives an input of the tomographic image;
a low motion site estimation unit that estimates a low motion site of the heart on the basis of the tomographic image;
an infarct site estimation unit that estimates an infarct site of the heart; and
a target site identification unit that identifies a site other than the infarct site among the low motion sites, as a target site.

15. An image processing method executed using an image processing apparatus, the method comprising:
an image input step of receiving, via a processor, as an input a tomographic image of a heart taken from outside a body;
a low motion site estimation step of estimating, via the processor, a low motion site of the heart on the basis of the tomographic image;
an infarct site estimation step of estimating, via the processor, an infarct site of the heart; and
a target site identification step of identifying, via the processor, a site other than the infarct site among the low motion sites, as a target site, the target site displayed on an output of the tomographic image.

16. The image processing method of claim 15, wherein estimating the infarct site of the heart comprises:
acquiring, via the processor, electrocardiographic information indicating an electrocardiogram of a heart wall with which a distal end portion of a catheter comes in contact via an electrode provided on the distal end portion of the catheter; and
estimating, via the processor, the infarct site on the basis of the acquired electrocardiographic information.

17. The image processing method of claim 15, wherein estimating the infarct site of the heart comprises:
acquiring, via the processor, electrocardiographic information indicating an electrocardiogram of a heart wall on the basis of a captured image obtained by imaging the heart by a predetermined imaging device; and
estimating, via the processor, the infarct site on the basis of the acquired electrocardiographic information.

18. The image processing method of claim 15, wherein the tomographic image is a first tomographic image, and wherein the method further comprises:
receiving, via the processor, an input of a second tomographic image of the heart taken from outside the body; and
estimating, via the processor, the infarct site based on the second tomographic image.

19. The image processing method of claim 18, further comprising:
receiving, via the processor, an input of a plurality of first tomographic images captured every predetermined time, wherein the low motion site is estimated based on temporal changes in the plurality of first tomographic images received.

20. The image processing method of claim 19, wherein in the target site identification step, the method further comprises:
selecting, via the processor, a first tomographic image corresponding to an expansion/contraction state of the heart in the second tomographic image from among the plurality of first tomographic images; and
identifying, via the processor, the target site using the selected first tomographic image.
